(19) United States
(12) Patent Application Publication
(10) Pub. No.: US 2017/0079616 A1
(43) Pub. Date: Mar. 23, 2017

(54) DIAGNOSTIC IMAGING CATHETER

(71) Applicant: TERUMO KABUSHIKI KAISHA, Tokyo (JP)

(72) Inventor: Keiichiro YAMAMOTO, Hiratsuka-city (JP)

(73) Assignee: TERUMO KABUSHIKI KAISHA, Tokyo (JP)

(21) Appl. No.: 15/264,986

(22) Filed: Sep. 14, 2016

(30) Foreign Application Priority Data
Sep. 18, 2015 (JP) 2015-186020

(51) Int. Cl. A61B 8/00; A61B 8/08; A61B 8/12

(52) U.S. Cl. CPC A61B 8/445; A61B 8/12; A61B 8/4494; A61B 8/0891

(57) ABSTRACT

A diagnostic imaging catheter is disclosed, which includes a sheath that is inserted into a body-cavity in a living body; an ultrasound transducer that is inserted into the sheath and is able to transmit and receive an ultrasound wave; a connector unit that is provided in a proximal portion of the sheath, an electrode terminal that is disposed inside the connector unit and is electrically connected to an external electrode terminal included in an external apparatus which transmits and receives an electrical signal with respect to the ultrasound transducer; a conductive member that is provided inside the connector unit, is electrically connected to a positive electrode and a negative electrode of the electrode terminal, and causes both the electrodes to be in a short-circuit state; and a switch unit that is configured to be able to cancel the short-circuit state of both the electrodes caused by the conductive member.

### CROSS-REFERENCES TO RELATED APPLICATIONS

This application claims priority to Japanese Application No. 2015-186020 filed on Sep. 18, 2015, the entire content of which is incorporated herein by reference.

### TECHNICAL FIELD

The present disclosure relates to a diagnostic imaging catheter.

### BACKGROUND DISCUSSION

In the related art, as diagnostic imaging catheters obtaining tomographic images of a blood vessel, there have been catheters which obtain images through an intra-vascular ultrasound (IVUS) diagnostic method.

The diagnostic imaging catheter used in the intra-vascular ultrasound diagnostic method has a configuration in which radial scanning is performed inside a blood vessel by using a probe internally equipped with an ultrasound transducer, processing such as amplification and wave detection is performed after a reflected wave (an ultrasound wave echo) reflected by biological tissue inside a body-cavity is received by the ultrasound transducer, and a cross-sectional image (a diagnostic image) of the blood vessel is depicted based on intensity of a generated ultrasound wave echo (refer to JP-A-2015-119994).

Generally, an ultrasound transducer is configured to be formed of a piezoelectric material having multiple crystals with spontaneous polarization. Since directions of spontaneous polarization of the crystals are different from each other, in order to obtain a piezoelectric effect, “polarization” in which an electric field is added and the directions of spontaneous polarization of the crystals are aligned so as to be in the same direction in a manufacturing process is performed.

If a polarized ultrasound transducer is kept in a not-in-use state, that is, a non-electrification state, there are cases where depolarization occurs due to an external factor such as a temperature and a residual strain generated when a piezoelectric material is processed. When such depolarization occurs, the piezoelectric effect is reduced, and thus, performance of the ultrasound transducer converting electrical energy into mechanical energy is degraded or reduced.

### SUMMARY

The present disclosure has been made in consideration of the aforementioned problem, and provides a diagnostic imaging catheter in which depolarization of an ultrasound transducer in a not-in-use state is suppressed and deterioration in quality of a diagnostic image can be prevented.

A diagnostic imaging catheter is disclosed, which includes a sheath that is inserted into a body-cavity in a living body; an ultrasound transducer that is inserted into the sheath and is able to transmit and receive an ultrasound wave; a connector unit that is provided in a proximal portion of the sheath; an electrode terminal that is disposed inside the connector unit and is electrically connected to an external electrode terminal included in an external apparatus which transmits and receives an electrical signal with respect to the ultrasound transducer; a conductive member that is provided inside the connector unit, is electrically connected to a positive electrode and a negative electrode of the electrode terminal, and causes both the electrodes to be in a short-circuit state; and a switch unit that is configured to be able to cancel the short-circuit state of both the electrodes caused by the conductive member, in response to connection between the electrode terminal and the external electrode terminal.

A diagnostic imaging catheter is disclosed, which includes a sheath; an ultrasound transducer that is inserted into the sheath and is configured to transmit and receive an ultrasound wave; a connector unit arranged in a proximal portion of the sheath; an electrode terminal disposed inside the connector unit and configured to e electrically connected to an external electrode terminal, which transmits and receives an electrical signal with respect to the ultrasound transducer; a conductive member inside the connector unit, the conductive member electrically connected to a positive electrode and a negative electrode of the electrode terminal, and which causes both the positive and the negative electrodes to be in a short-circuit state; and a switch unit that is configured to cancel the short-circuit state of both the positive and the negative electrodes caused by the conductive member, in response to connection between the electrode terminal and the external electrode terminal.

A method is disclosed comprising: inserting a diagnostic catheter into a blood vessel of a human body, the diagnostic catheter including a sheath that is inserted into a body-cavity in a living body, an ultrasound transducer that is inserted into the sheath and is able to transmit and receive an ultrasound wave, a connector unit that is provided in a proximal portion of the sheath, an electrode terminal that is disposed inside the connector unit and is electrically connected to an external electrode terminal included in an external apparatus which transmits and receives an electrical signal with respect to the ultrasound transducer, a conductive member that is provided inside the connector unit, is electrically connected to a positive electrode and a negative electrode of the electrode terminal, and causes both the electrodes to be in a short-circuit state, and a switch unit that is configured to be able to cancel the short-circuit state of both the electrodes caused by the conductive member, in response to connection between the electrode terminal and the external electrode terminal.

According to the diagnostic imaging catheter having the above-described configuration, in a not-in-use state before the external apparatus is connected to the diagnostic imaging catheter for use, the positive electrode and the negative electrode of the electrode terminal electrically connected to the ultrasound transducer can be kept in a short-circuit state. Therefore, depolarization of the ultrasound transducer is suppressed, and a piezoelectric effect from polarization can be retained. Accordingly, degradation of performance of the ultrasound transducer can be suppressed, and deterioration in quality of a diagnostic image obtained through the diagnostic imaging catheter can be prevented.

### BRIEF DESCRIPTION OF THE DRAWINGS

FIG. 1 is a plan view schematically illustrating a diagnostic imaging catheter, according to an embodiment of the present disclosure.

FIGS. 2A and 2B are views schematically illustrating an overall configuration of the diagnostic imaging catheter, according to the embodiment, wherein FIG. 2A is a side view of the diagnostic imaging catheter before executing a pull-back operation (an evacuating operation), and FIG. 2B is a side view of the diagnostic imaging catheter when the pull-back operation is executed.

FIGS. 3A and 3B are views illustrating a configuration of each of units in the diagnostic imaging catheter, according to the embodiment, wherein FIG. 3A is an enlarged sectional view illustrating a configuration of the diagnostic imaging catheter on the distal side, and FIG. 3B is an enlarged sectional view illustrating a configuration of the diagnostic imaging catheter on the proximal side (hand-side).

FIGS. 4A and 4B are enlarged sectional views of a connector unit according to the embodiment, wherein FIG. 4A illustrates a state before an external electrode terminal of an external apparatus is connected, and FIG. 4B illustrates a state after the external electrode terminal is connected.

FIGS. 5A and 5B are enlarged sectional views of an electrode terminal according to the embodiment, wherein FIG. 5A illustrates a state before the external electrode terminal is connected, and FIG. 5B illustrates a state after the external electrode terminal is connected.

FIGS. 6A and 6B are enlarged sectional views of an electrode terminal according to Modification Example 1, wherein FIG. 6A illustrates a state before the external electrode terminal is connected, and FIG. 6B illustrates a state after the external electrode terminal is connected.

FIGS. 7A-7C are schematic views of an electrode terminal according to Modification Example 2, wherein FIG. 7A is an enlarged plan view of the electrode terminal viewed in an insertion direction of the external electrode terminal, FIG. 7B is an enlarged sectional view illustrating the electrode terminal in a state before the external electrode terminal is connected, and FIG. 7C is an enlarged sectional view illustrating the electrode terminal in a state after the external electrode terminal is connected.

### DETAILED DESCRIPTION

Hereinafter, with reference to the accompanying drawings, an embodiment of the present disclosure will be described. Note that, the description below does not limit the meanings of the technical scope and the terms disclosed in Claims. In addition, for the convenience of description, there are cases where the dimensional ratios of the drawings are exaggerated and are different from the actual ratios.

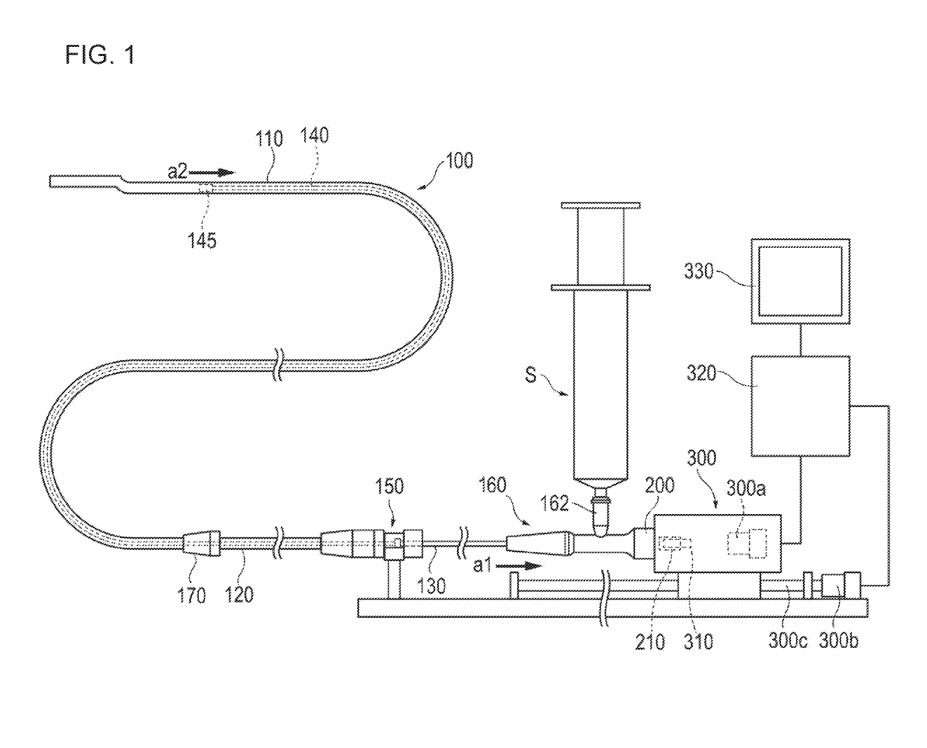

With reference to FIGS. 1 to 5B, the diagnostic imaging catheter 100 will be described. The diagnostic imaging catheter 100 according to the present embodiment is a diagnostic imaging catheter which is used in an intra-vascular ultrasound diagnostic method.

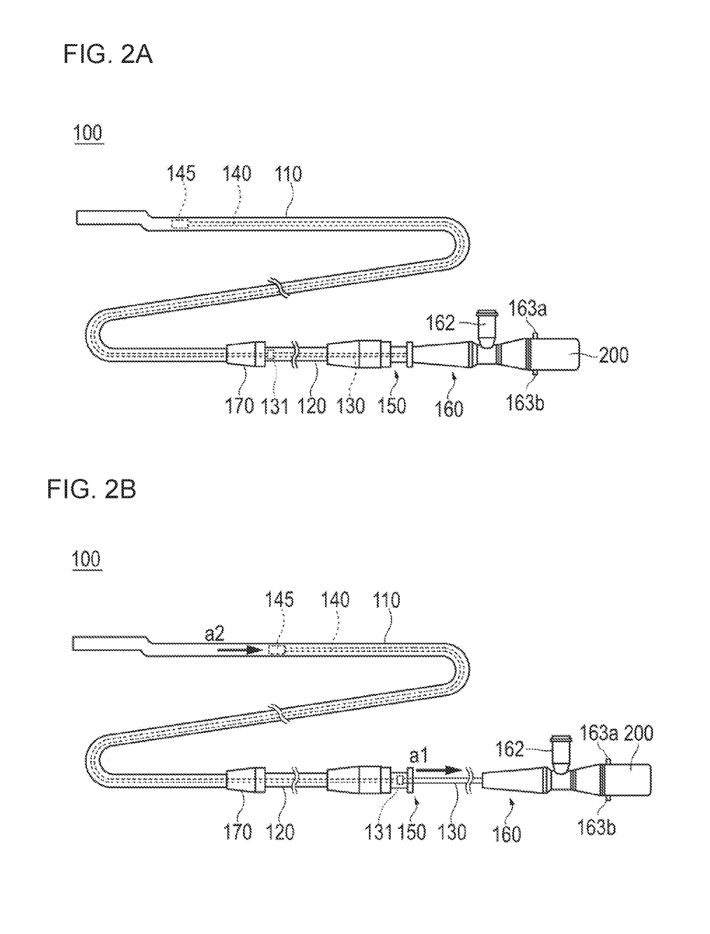

As illustrated in FIGS. 1, 2A, and 2B, generally, the diagnostic imaging catheter 100 can include a sheath 110 that is inserted into a body-cavity in a living body, an outer tube 120 that is provided on the proximal side of the sheath 110, an inner shaft 130 that is inserted into the outer tube 120 so as to be movable forward and rearward, a drive shaft 140 that has a signal transmitting and receiving unit 145 transmitting and receiving a signal at the distal end and is rotatably provided inside the sheath 110, a unit connector 150 that is configured to be provided on the proximal side of the outer tube 120 and to accept the inner shaft 130, and a hub 160 that is provided on the proximal side of the inner shaft 130.

In description of the specification, a side of the diagnostic imaging catheter 100 inserted into a body-cavity will be referred to as the distal end or the distal side, a side of the hub 160 provided in the diagnostic imaging catheter 100 will be referred to as the proximal end or the proximal side, and the extending direction of the sheath 110 will be referred to as the axial direction.

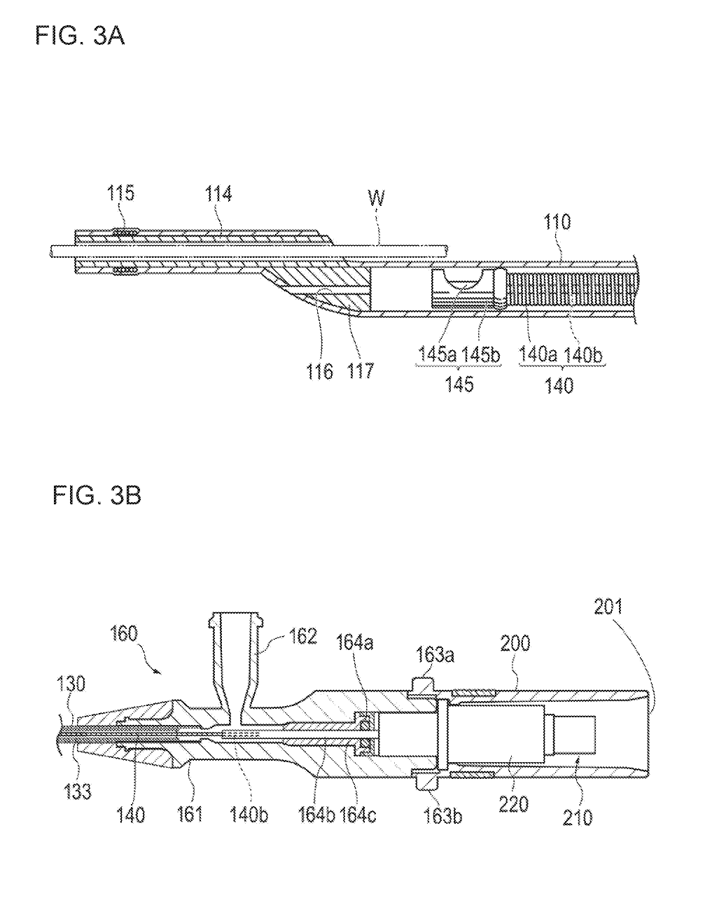

As illustrated in FIGS. 2A and 3B, the drive shaft 140 passes through the sheath 110, the outer tube 120 connected to the proximal end of the sheath 110, and the inner shaft 130 inserted into the outer tube 120, and extends to the inside of the hub 160.

The hub 160, the inner shaft 130, the drive shaft 140, and the signal transmitting and receiving unit 145 are connected to each other so as to integrally move forward and rearward in the axial direction. Therefore, for example, when the hub 160 is operated so as to be pushed toward the distal side, the inner shaft 130 connected to the hub 160 is thrust into the outer tube 120 and the unit connector 150, and the drive shaft 140 and the signal transmitting and receiving unit 145 move inside the sheath 110 toward the distal side. For example, when the hub 160 is operated so as to be pulled toward the proximal side, as indicated with the arrow a1 in FIGS. 1 and 2B, the inner shaft 130 is drawn out through the outer tube 120 and the unit connector 150, and as indicated with the arrow a2, the drive shaft 140 and the signal transmitting and receiving unit 145 move inside the sheath 110 toward the proximal side.

As illustrated in FIG. 2A, when the inner shaft 130 is thrust toward the distal side to the end, the distal portion of the inner shaft 130 reaches the vicinity of a relay connector 170. In this case, the signal transmitting and receiving unit 145 is positioned in the vicinity of the distal end of the sheath 110. The relay connector 170 is a connector which causes the sheath 110 and the outer tube 120 to be connected to each other.

As illustrated in FIG. 2B, a coming-off prevention connector 131 is provided at the distal end of the inner shaft 130. The coming-off prevention connector 131 has a function of preventing the inner shaft 130 from coming off from the outer tube 120. The coming-off prevention connector 131 is configured to be caught at a predetermined position in the inner wall of the unit connector 150 when the hub 160 is pulled toward the proximal side to the end, for example, when the inner shaft 130 is drawn out to the end through the outer tube 120 and the unit connector 150. Note that, in order to prevent the inner shaft 130 from coming off from the outer tube 120, the coming-off prevention connector 131 is not necessarily provided. For example, the inner shaft 130 may be prevented from coming off from the outer tube 120 by processing the distal end of the inner shaft 130 such that the inner shaft 130 does not come off from the outer tube 120.

As illustrated in FIG. 3A, the drive shaft 140 can include an elastic pipe body 140a and a signal wire 140b which is inserted through the inside of the pipe body 140a. For example, the pipe body 140a can be configured to be a coil having multiple layers of which winding directions around the axis are different from each other. As a configuration material of the coil, for example, stainless steel and a nickel-titanium (Ni—Ti) alloy can be used. For example, the signal wire 140b can be configured to be a twist pair cable or a coaxial cable.

The signal transmitting and receiving unit 145 has an ultrasound transducer 145a transmitting and receiving an ultrasound wave, and a housing 145b in which the ultrasound transducer 145a is housed.

The ultrasound transducer 145a has a function of transmitting an ultrasound wave as an inspection wave into a body-cavity and receiving the ultrasound wave reflected from the body-cavity. The ultrasound transducer 145a can be formed of a material known as a piezoelectric material. As a material configuring the ultrasound transducer 145a, for example, ceramics or crystal can be used.

The diagnostic imaging catheter 100 is shipped in a state where the ultrasound transducer 145a is subjected to polarization processing during the manufacturing process. When a voltage is applied to the polarized ultrasound transducer 145a, an ultrasound wave is generated due to a piezoelectric effect. The ultrasound transducer 145a is electrically connected to the below-described electrode terminal 210 via the signal wire 140b.

A priming liquid discharge member 117 having a priming liquid discharge hole 116 for discharging priming liquid formed therein is installed at the distal portion of the sheath 110. When the diagnostic imaging catheter 100 is in use, in order to reduce attenuation of ultrasound waves caused by air inside the sheath 110 and to efficiently transmit and receive the ultrasound waves, the inside of the sheath 110 is filled with the priming liquid. When the sheath 110 is filled with the priming liquid, gas such as air staying inside the sheath 110 can be discharged through the priming liquid discharge hole 116 formed in the priming liquid discharge member 117.

In accordance with an exemplary embodiment, the sheath 110 is formed of a material having high transmittance of an ultrasound wave. A range in which the ultrasound transducer 145a moves in the axial direction of the sheath 110 (the distal portion of the sheath 110) is configured to be an acoustic window portion of which transmittance of an ultrasound wave is formed to be higher than other portions.

The sheath 110 is formed of an elastic material, and the material is not particularly limited. For example, various types of thermoplastic elastomers can be used for the sheath 110 such as a styrene-based elastomer, a polyolefin-based elastomer, a polyurethane-based elastomer, a polyester-based elastomer, a polyimide-based elastomer, a polyimide-based elastomer, a polybutadiene-based elastomer, a trans-polyisoprene-based elastomer, a fluororubber-based elastomer, and a chlorinated polyethylene-based elastomer. One type or a combination of two or more types among thereof (a polymer alloy, a polymer blend, and a laminated body) can also be adopted. Note that, a hydrophilic lubrication coating layer exhibiting lubricity at the time of wetting can be disposed on the outer surface of the sheath 110.

A guide wire insertion member 114 provided with a lumen through which a guide wire W can be inserted is attached to the distal portion of the sheath 110. In addition, the guide wire insertion member 114 is provided with a marker 115 having X-ray contrast properties.

As illustrated in FIG. 3B, the hub 160 has a hollow-shaped hub main body 161, the connector unit 200 in which the electrode terminal 210 mechanically and electrically connected to the below-described external apparatus 300 is disposed, a port 162 which communicates with the inside of the hub main body 161, direction checking spurs 163a and 163b for checking the orientation of the hub 160 when performing connection with respect to the external apparatus 300, a seal member 164a which seals a place on the proximal side closer than the port 162, a connection pipe 164b which holds the drive shaft 140, and a bearing 164c which rotatably supports the connection pipe 164b.

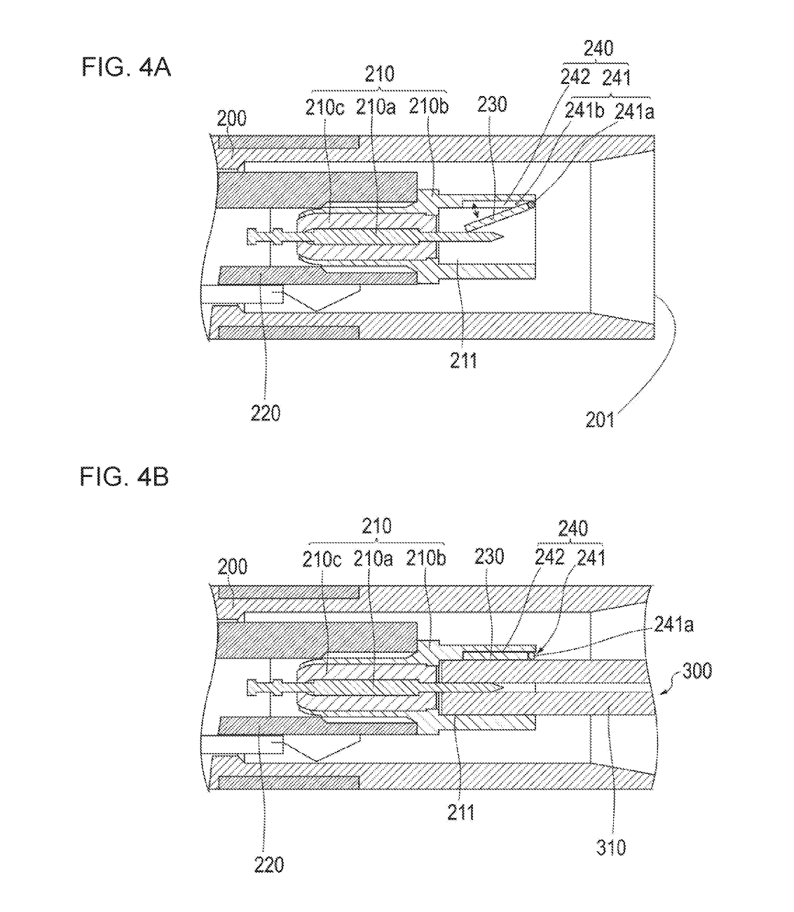

As illustrated in FIG. 4A, the electrode terminal 210 and a rotor 220 are disposed inside the connector unit 200. A circular opening portion 201 is formed at the proximal end of the connector unit 200. As illustrated in FIG. 4B, an external electrode terminal 310 included in the external apparatus 300 (refer to FIG. 1) passes through the opening portion 201 of the connector unit 200 and is inserted into the connector unit 200.

The electrode terminal 210 is configured to be a coaxial receptacle (male connector unit). Meanwhile, the external electrode terminal 310 included in the external apparatus 300 is configured to be a cylindrical coaxial plug (female connector unit). The electrode terminal 210 and the external electrode terminal 310 are electrically connected to each other when the coaxial receptacle is accepted by the coaxial plug.

As illustrated in FIG. 4A, the electrode terminal 210 has a cylindrical negative electrode 210b, a positive electrode 210a which extends along the central axis of the negative electrode 210b, and an insulation member 210c which is provided between the positive electrode 210a and the negative electrode 210b and isolates the positive electrode 210a and the negative electrode 210b from each other. A fitting groove 211 in which the external electrode terminal 310 is put and fitted is formed between the positive electrode 210a and the negative electrode 210b.

As illustrated in FIGS. 4A and 4B, the diagnostic imaging catheter 100 has a conductive member 230 which is disposed in the connector unit 200 and causes both the electrodes 210a and 210b to be in a short-circuit state by being electrically connected to the positive electrode 210a and the negative electrode 210b of the electrode terminal 210, and a switch unit 240 which is configured to be able to cancel the short-circuit state of both the electrodes 210a and 210b caused by the conductive member 230, in response to connection between the electrode terminal 210 and the external electrode terminal 310.

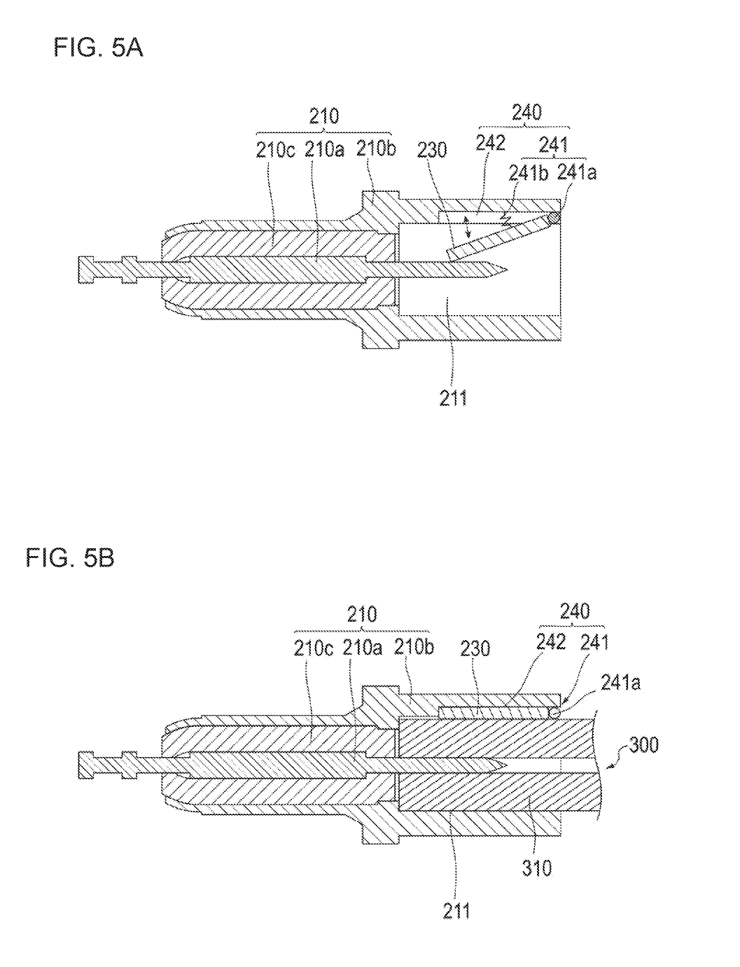

As illustrated in FIG. 5A, the conductive member 230 is configured to be a rod-like member which is disposed in the fitting groove 211 of the electrode terminal 210 and extends from the proximal side of the negative electrode 210b toward the distal side.

The conductive member 230 can be configured to be formed of a material having electric resistivity as low to the extent as the positive electrode 210a and the negative electrode 210b can be in a short-circuit state in response to connection to the electrode terminal 210. As a material configuring the conductive member 230, for example, a metal material such as copper and aluminum can be used.

The switch unit 240 has a biasing member 241 which supports the conductive member 230 in a movable state in a direction intersecting the extending direction of the electrode terminal 210 (direction indicated with the arrow in the diagram) and applies biasing force in a direction approaching the positive electrode 210a side to the conductive member 230, and an accommodation space 242 which can accommodate the conductive member 230 and the biasing member 241.

The biasing member 241 according to the present embodiment has a support portion 241a which is fixed to the inner wall of the negative electrode 210b on the proximal side and turnably supports the proximal side of the conductive member 230, and a spring 241b which is provided between the inner wall of the negative electrode 210b and the conductive member 230. In accordance with an exemplary embodiment, for example, the biasing member 241 is not limited to the above-described configuration and can be configured to be a torsion spring or a spring hinge.

As the conductive member 230 rotates around the support portion 241a serving as the rotary center, the distal side of the conductive member 230 comes close to and is separated from the inner wall of the negative electrode 210b. In a case where the external electrode terminal 310 is not connected to the electrode terminal 210, as illustrated in FIG. 5A, the distal side of the conductive member 230 comes into contact with the positive electrode 210a due to biasing force of the spring 241b.

In the present embodiment, similar to the conductive member 230, the support portion 241a is formed of a conductive material. In addition, the support portion 241a is interlocked to the inner wall of the negative electrode 210b and the proximal side of the conductive member 230 through a method of being able to be electrically connected thereto. The interlocking method is not particularly limited. For example, soldering and a conductive adhesive can be adopted. Therefore, in a state where the conductive member 230 is in contact with the positive electrode 210a, the positive electrode 210a and the negative electrode 210b are electrically connected to each other via the conductive member 230 and the support portion 241a, thereby being in a short-circuit state.

The accommodation space 242 is formed by providing a recessed portion caved in the inner wall of the negative electrode 210b. The accommodation space 242 extends from the proximal side of the negative electrode 210b toward the distal side. The support portion 241a is fixed to the inner wall of the negative electrode 210b on the proximal side of the accommodation space 242.

As illustrated in FIG. 5B, when the external electrode terminal 310 is inserted into the fitting groove 211, the conductive member 230 is thrust into the accommodation space 242 against the biasing force of the biasing member 241 while rotating around the support portion 241a serving as the rotary center. Accordingly, the positive electrode 210a and the conductive member 230 are separated from each other, and thus, the short-circuit state of both the electrodes 210a and 210b is cancelled. In this specification, hereinafter, a state where the short-circuit state is cancelled will be referred to as “the non-short-circuit state”.

When the external electrode terminal 310 is detached from the electrode terminal 210, as illustrated in FIG. 5A, due to biasing force of the biasing member 241, the distal side of the conductive member 230 rotates toward the negative electrode 210b, and the conductive member 230 and the positive electrode 210a come into contact with each other again. In this manner, the switch unit 240 according to the present embodiment can reversibly switch both the electrodes 210a and 210b between a short-circuit state and a non-short-circuit state in response to connection and detachment of the external electrode terminal 310 with respect to the electrode terminal 210.

As illustrated in FIG. 3B, the rotor 220 holds the connection pipe 164b in a non-rotatable manner and rotates integrally with the electrode terminal 210.

As illustrated in FIG. 3B, the inner shaft 130 is connected to the distal portion of the hub main body 161. The drive shaft 140 is drawn out through the inner shaft 130 inside the hub main body 161. A protection tube 133 is disposed between the inner shaft 130 and the drive shaft 140. The protection tube 133 has a function of pressing vibration (flapping) of the drive shaft 140 caused by the clearance generated at the time of pulling-back.

In order to transfer rotations of the rotor 220 to the drive shaft 140, the connection pipe 164b holds the drive shaft 140 at the end portion on a side opposite to the rotor 220 (the distal end of the connection pipe 164b). The signal wire 140b (refer to FIG. 3A) is inserted through the inside of the connection pipe 164b. One end of the signal wire 140b is connected to the electrode terminal 210, and the other end passes through the inside of the drive shaft 140 and is connected to the ultrasound transducer 145a. A signal received by the ultrasound transducer 145a is transmitted to the external apparatus 300 via the electrode terminal 210 and is subjected to predetermined processing, thereby being displayed as an image.

Repeatedly with reference to FIG. 1, the diagnostic imaging catheter 100 is driven by being connected to the external apparatus 300.

The external apparatus 300 is connected to the connector unit 200 provided on the proximal side of the hub 160. As described above, the external apparatus 300 has the external electrode terminal 310. The external electrode terminal 310 is electrically connected to the electrode terminal 210 included in the diagnostic imaging catheter 100.

In addition, the external apparatus 300 has a motor 300a which is a power source for rotating the drive shaft 140, and a motor 300b which is a power source for moving the drive shaft 140 in the axial direction. A rotary motion of the motor 300b is converted into a motion in the axial direction by a ball screw 300c connected to the motor 300b.

An operation of the external apparatus 300 is controlled by a control apparatus 320 which is electrically connected thereto. The control apparatus 320 includes a central processing unit (CPU) and a memory as main configuration elements. The control apparatus 320 is electrically connected to a monitor 330.

Subsequently, an example of use of the diagnostic imaging catheter 100 will be described.

As illustrated in FIG. 5A, in a not-in-use state before the external electrode terminal 310 is connected to the electrode terminal 210, due to biasing force of the biasing member 241, the distal side of the conductive member 230 is retained in a state of being in contact with the positive electrode 210a. Therefore, the positive electrode 210a and the negative electrode 210b are electrically connected to each other via the conductive member 230 and the biasing member 241, thereby being in a short-circuit state.

Subsequently, when the external electrode terminal 310 of the external apparatus 300 is connected to the electrode terminal 210 of the diagnostic imaging catheter 100, the conductive member 230 is thrust into the accommodation space 242, and the positive electrode 210a and the conductive member 230 are separated from each other. Accordingly, the short-circuit state of both the electrodes 210a and 210b can be cancelled. As a result thereof, the electrode terminal 210 and the external electrode terminal 310 are electrified, and the ultrasound transducer 145a is in a state of being able to conduct a function of transmitting and receiving an ultrasound wave. In this manner, the positive electrode 210a and the negative electrode 210b can be switched from a short-circuit state to a non-short-circuit state by only connecting the external electrode terminal 310 to the electrode terminal 210. Therefore, both the electrodes 210a and 210b can be easily switched from a short-circuit state to a non-short-circuit state and can be reliably prevented from being forgotten to be switched from a short-circuit state to a non-short-circuit state.

Thereafter, as illustrated in FIG. 1, a user connects a syringe S having the priming liquid therein to the port 162 and fills the sheath 110 with the priming liquid by pressing a plunger of the syringe S.

After performing priming, as illustrated in FIG. 2A, the user thrusts the hub 160 until the hub 160 abuts on the proximal end of the unit connector 150 and moves the signal transmitting and receiving unit 145 to the distal side. In this state, the sheath 110 is inserted into a body-cavity (for example, a blood vessel) along the guide wire W toward a target position.

When a tomographic image is obtained at a target position inside a body-cavity, as illustrated in FIG. 2B, the signal transmitting and receiving unit 145 transmits and receives an ultrasound wave while moving together with the drive shaft 140 toward the proximal side. In addition, in this case, the signal transmitting and receiving unit 145 rotates together with the drive shaft 140.

The control apparatus 320 controls the motor 300a illustrated in FIG. 1 and controls rotation around the axis of the drive shaft 140. In addition, the control apparatus 320 controls the motor 300b and controls movement of the drive shaft 140 in the axial direction.

The signal transmitting and receiving unit 145 transmits an ultrasound wave to the inside of a body based on a signal sent from the control apparatus 320. A signal received by the signal transmitting and receiving unit 145 and corresponding to a reflected wave is sent to the control apparatus 320 via the drive shaft 140 and the external apparatus 300. The control apparatus 320 generates a tomographic image of a body-cavity based on a signal sent from the signal transmitting and receiving unit 145 and causes the monitor 330 to display the generated image.

The connector unit 200 provided inside the hub 160 rotates in a state of being connected to the external apparatus 300, and the drive shaft 140 rotates in association therewith. The rotating speed of the connector unit 200 and the drive shaft 140 can be 1,800 rpm, for example.

As described above, the diagnostic imaging catheter 100 according to the present embodiment can include the sheath 110 that is inserted into a body-cavity in a living body; the ultrasound transducer 145a that is inserted into the sheath 110 and is able to transmit and receive an ultrasound wave; the connector unit 200 that is provided in a proximal portion of the sheath 110; the electrode terminal 210 that is disposed inside the connector unit 200 and is electrically connected to the external electrode terminal 310 included in the external apparatus 300 which transmits and receives an electrical signal with respect to the ultrasound transducer 145a; the conductive member 230 that is provided inside the connector unit 200, is electrically connected to the positive electrode 210a and the negative electrode 210b of the electrode terminal 210, and causes both the electrodes 210a and 210b to be in a short-circuit state; and the switch unit 240 that is configured to be able to cancel the short-circuit state of both the electrodes 210a and 210b caused by the conductive member 230, in response to connection between the electrode terminal 210 and the external electrode terminal 310.

According to the diagnostic imaging catheter 100 having such a configuration, in a not-in-use state before the diagnostic imaging catheter 100 is connected to the external apparatus 300 for use, the positive electrode 210a and the negative electrode 210b of the electrode terminal 210 electrically connected to the ultrasound transducer 145a can be kept in a short-circuit state. Therefore, depolarization of the ultrasound transducer 145a can be suppressed, and a piezoelectric effect from polarization can be retained. Accordingly, degradation of performance of the ultrasound transducer 145a can be suppressed, and deterioration in quality of a diagnostic image obtained through the diagnostic imaging catheter 100 can be prevented.

In addition, the switch unit 240 has the biasing member 241 which applies biasing force in a direction approaching the positive electrode 210a to the conductive member 230. The switch unit 240 is configured to be able to cancel the short-circuit state of both the electrodes 210a and 210b as the conductive member 230 is separated from the positive electrode 210a against the biasing force of the biasing member 241 when the external electrode terminal 310 is inserted into the electrode terminal 210. Therefore, both the electrodes 210a and 210b can be reversibly switched between a short-circuit state and a non-short-circuit state in response to connection and detachment of the external electrode terminal 310 with respect to the electrode terminal 210.

In addition, the biasing member 241 supports the conductive member 230 in a movable state in a direction intersecting the extending direction of the electrode terminal 210. Therefore, the conductive member 230 can be moved in a direction intersecting the extending direction of the electrode terminal 210 by inserting the external electrode terminal 310 into the electrode terminal 210, and both the electrodes 210a and 210b can be easily switched from a short-circuit state to a non-short-circuit state by separating the positive electrode 210a and the conductive member 230 from each other.

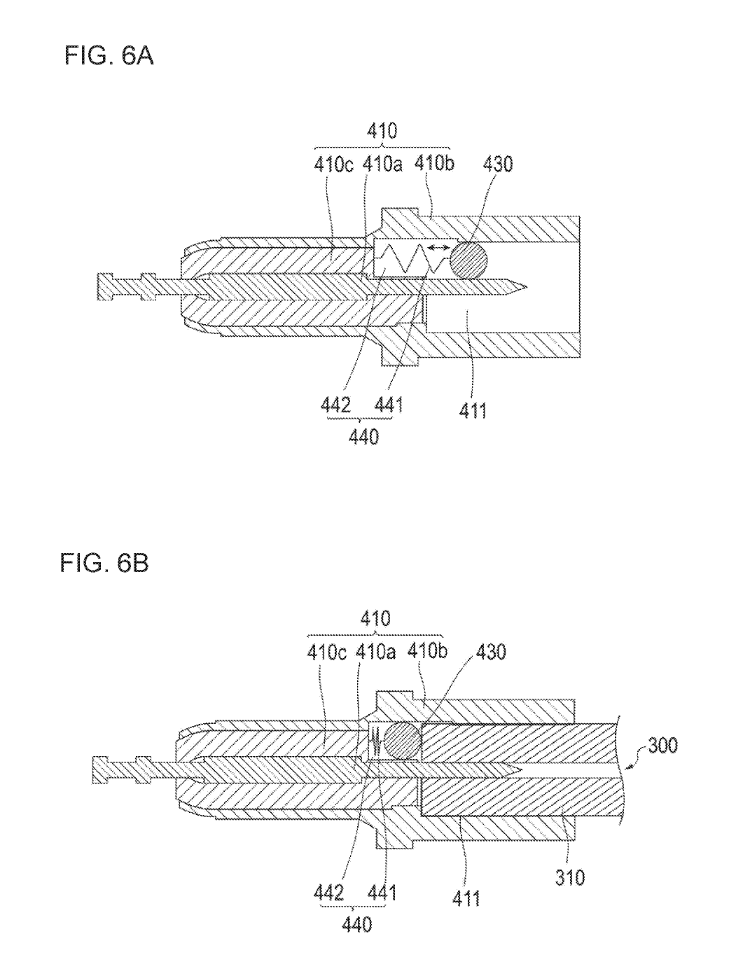

FIGS. 6A and 6B are enlarged sectional view of an electrode terminal 410 according to Modification Example 1 of the above-described embodiment. FIG. 6A illustrates a state before the external electrode terminal 310 is connected, and FIG. 6B illustrates a state after the external electrode terminal 310 is connected.

As illustrated in FIG. 6A, the diagnostic imaging catheter 100 according to Modification Example 1 is different from that in the above-described embodiment in that a conductive member 430 is supported by a biasing member 441 in a movable state in the extending direction of the electrode terminal 410 (the direction indicated with the arrow in the diagram). Hereinafter, the diagnostic imaging catheter 100 according to Modification Example 1 will be described in detail. Note that, the same reference numerals and signs will be applied to the configurations similar to those in the above-described embodiment, and description thereof will be omitted.

The electrode terminal 410 has a cylindrical negative electrode 410b, a positive electrode 410a which extends along the central axis of the negative electrode 410b, and an insulation member 410c which is provided between the positive electrode 410a and the negative electrode 410b and isolates the positive electrode 410a and the negative electrode 410b from each other. A fitting groove 411 in which the external electrode terminal 310 is put and fitted is formed between the positive electrode 410a and the negative electrode 410b.

The conductive member 430 is configured to be a spherical member. The conductive member 430 is disposed in the fitting groove 411 of the electrode terminal 410. The diameter of the conductive member 430 can be set to a length such that the conductive member 430 can come into contact with the positive electrode 410a and the negative electrode 410b and can slide between the positive electrode 410a and the negative electrode 410b. In this manner, when the outer shape of the conductive member 430 is formed to be a spherical shape, the conductive member 430 can easily slide between the positive electrode 410a and the negative electrode 410b. However, the outer shape of the conductive member 430 is not limited to the spherical shape as long as the conductive member 430 can come into contact with the positive electrode 410a and the negative electrode 410b and can slide between the positive electrode 410a and the negative electrode 410b. For example, the outer shape may be a quadratic prism or a polyhedron.

The conductive member 430 can be configured to be formed of a material having electric resistivity as low to the extent as the positive electrode 410a and the negative electrode 410b can be in a short-circuit state in response to connection to the electrode terminal 410. As a material configuring the conductive member 430, for example, a metal material such as copper and aluminum can be used.

A switch unit 440 has the biasing member 441 which supports the conductive member 430 in a movable state in the extending direction of the electrode terminal 410 and applies biasing force in a direction approaching the positive electrode 410a side to the conductive member 430, and an accommodation space 442 which can accommodate the conductive member 430 and the biasing member 441.

The biasing member 441 according to the present embodiment is configured to be a spring which is flexible in the extending direction of the electrode terminal 410 in the fitting groove 411. Note that, the biasing member 441 is not limited to the spring. For example, an elastic member such as a sponge can be adopted.

The proximal side of the biasing member 441 is fixed to the distal side of the insulation member 410c, and the distal side of the biasing member 441 is fixed to the conductive member 430. Therefore, the conductive member 430 is configured to be slidable between the positive electrode 410a and the negative electrode 410b due to flexibility of the biasing member 441. In a case where the external electrode terminal 310 is not connected to the electrode terminal 410, as illustrated in FIG. 6A, the conductive member 430 is disposed at a position where the conductive member 430 comes into contact with the positive electrode 410a and the negative electrode 410b due to biasing force of the biasing member 441. Therefore, the positive electrode 410a and the negative electrode 410b are electrically connected to each other via the conductive member 430, thereby being in a short-circuit state.

The accommodation space 442 is formed by providing a recessed portion caved in the inner wall of the negative electrode 410b and the insulation member 410c. The biasing member 441 is fixed to the insulation member 410c on the proximal side of the accommodation space 442. In accordance with an exemplary embodiment, the accommodation space 442 is acceptable as long as the conductive member 430 disposed in the accommodation space 442 is configured not to come into contact with at least any one of the positive electrode 410a and the negative electrode 410b. Therefore, for example, the accommodation space 442 may be formed by providing the recessed portion caved in the inner wall of the positive electrode 410a and the insulation member 410c or may be formed by providing the recessed portion caved in only the inner wall of the insulation member 410c.

As illustrated in FIG. 6B, when the external electrode terminal 310 is inserted into the fitting groove 411, the conductive member 430 is thrust into the accommodation space 442 against the biasing force of the biasing member 441. Since the conductive member 430 is isolated from the positive electrode 410a by the insulation member 410c, the positive electrode 410a and the negative electrode 410b are in a non-short-circuit state.

When the external electrode terminal 310 is detached from the electrode terminal 410, as illustrated in FIG. 6A, due to biasing force of the biasing member 441, the conductive member 430 moves to the proximal side of the fitting groove 411, the conductive member 430 and the positive electrode 410a come into contact with each other again, and the positive electrode 410a and the negative electrode 410b are in a short-circuit state. In this manner, the switch unit 440 according to the present embodiment can reversibly switch both the electrodes 410a and 410b between a short-circuit state and a non-short-circuit state in response to connection and detachment of the external electrode terminal 310 with respect to the electrode terminal 410.

As described above, according to the diagnostic imaging catheter 100 in Modification Example 1, the conductive member 430 is supported by the biasing member 441 in a movable state in the extending direction of the electrode terminal 410. Therefore, the conductive member 430 can be moved in the extending direction of the electrode terminal 410 by inserting the external electrode terminal 310 into the electrode terminal 410, and both the electrodes 410a and 410b can be easily switched from a short-circuit state to a non-short-circuit state by separating the positive electrode 410a from the conductive member 430.

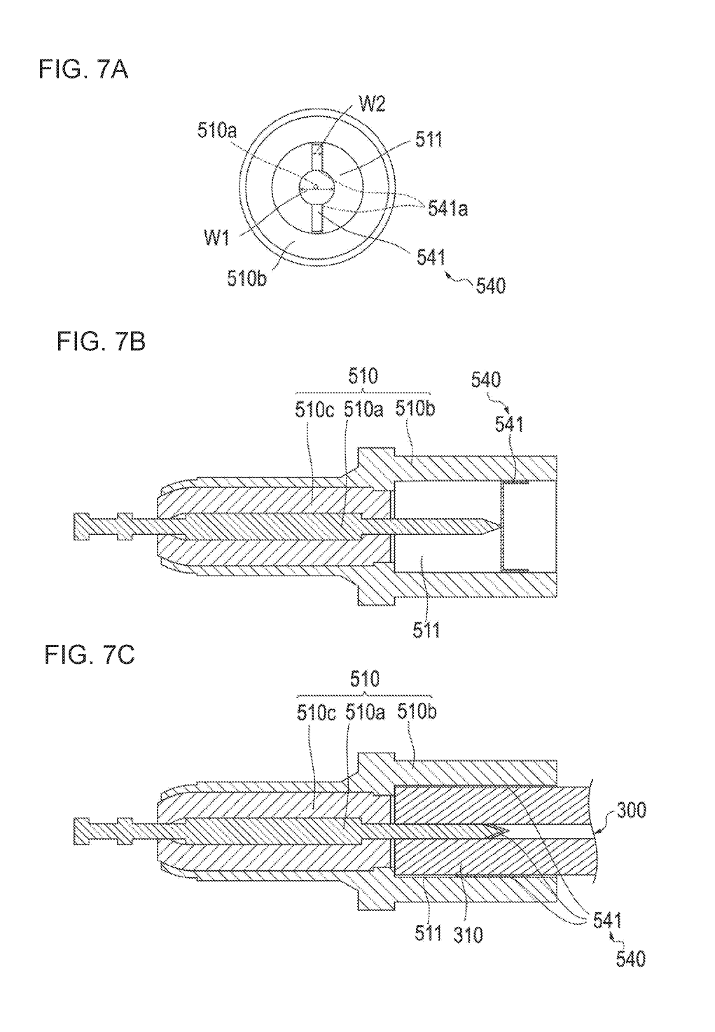

FIGS. 7A-7C are schematic views of an electrode terminal 510 according to Modification Example 2. FIG. 7A is an enlarged plan view of the electrode terminal 510 viewed in an insertion direction of the external electrode terminal 310, FIG. 7B is an enlarged sectional view illustrating the electrode terminal 510 in a state before the external electrode terminal 310 is connected, and FIG. 7C is an enlarged sectional view illustrating the electrode terminal 510 in a state after the external electrode terminal 310 is connected.

As illustrated in FIG. 7B, the diagnostic imaging catheter 100 according to Modification Example 2 is different from those in the above-described embodiment and Modification Example 1 in that a switch unit 540 has a retention member 541 which retains the short-circuit state of a positive electrode 510a and a negative electrode 510b in a state before the external electrode terminal 310 is inserted into the electrode terminal 510 and retains a state where the short-circuit state of both the electrodes 510a and 510b is cancelled in a state after the external electrode terminal 310 is detached from the electrode terminal 510. Hereinafter, the diagnostic imaging catheter 100 according to Modification Example 2 will be described in detail. Note that, the same reference numerals and signs will be applied to the configurations similar to those in the above-described embodiment and Modification Example 1, and description thereof will be omitted.

As illustrated in FIG. 7B, the electrode terminal 510 has a cylindrical negative electrode 510b, the positive electrode 510a which extends along the central axis of the negative electrode 510b, and an insulation member 510c which is provided between the positive electrode 510a and the negative electrode 510b and isolates the positive electrode 510a and the negative electrode 510b from each other. A fitting groove 511 in which the external electrode terminal 310 is put and fitted is formed between the positive electrode 510a and the negative electrode 510b.

The retention member 541 of the switch unit 540 is configured to be formed of a conductive sheet material. As the retention member 541, for example, a known conductive sheet material such as metal foil including copper foil and aluminum foil can be adopted.

The retention member 541 also functions as a conductive member by causing the positive electrode 510a and the negative electrode 510b to be electrically connected to each other and to be in a short-circuit state.

In the present embodiment, as illustrated in FIG. 7A, the retention member 541 has a shape extending in the radial direction of the negative electrode 510b. As illustrated in FIG. 7B, both ends of the retention member 541 are fixed to the inner wall of the negative electrode 510b. The fixing method is not particularly limited as long as the retention member 541 and the negative electrode 510b can be electrically connected to each other. For example, the fixing method can include soldering, a conductive adhesive, or a conductive adhesive tape. In addition, a central portion of the retention member 541 is in contact with the positive electrode 510a.

Note that, as illustrated in FIG. 7A, a width W1 of the central portion of the retention member 541 along a direction orthogonal to the extending direction of the retention member 541 may be formed so as to be longer than a width W2 on an end portion side. When the width W1 of the central portion is long, the central portion of the retention member 541 can be more reliably in contact with the positive electrode 510a. Meanwhile, the width W2 on the end portion side is shortened and the area of the retention member 541 is reduced. Accordingly, as described below, when the external electrode terminal 310 is inserted into the fitting groove 511, the retention member 541 can be reliably broken, and the broken retention member 541 is prevented from being interposed between the electrode terminal 510 and the external electrode terminal 310 in a state of being in contact with both the positive electrode 510a and the negative electrode 510b.

In accordance with an exemplary embodiment, the central portion of the retention member 541 may be fixed to the positive electrode 510a such that the central portion of the retention member 541 reliably comes into contact with the positive electrode 510a. The fixing method is not particularly limited as long as the retention member 541 and the positive electrode 510a can be electrically connected to each other. For example, the fixing method can include soldering or a conductive adhesive.

As illustrated in FIG. 7A, a breakage portion 541a can be provided between portions in contact with the positive electrode 510a and the negative electrode 510b in the retention member 541 so as to be easily broken when being brought into contact with the external electrode terminal 310. For example, the breakage portion 541a can be formed by providing notches in the retention member 541 at predetermined distances or can be formed by causing a portion of the retention member 541 to be thin in thickness.

As illustrated in FIG. 7C, the retention member 541 is broken by inserting the external electrode terminal 310 into the fitting groove 511. Since electrical connection between the positive electrode 510a and the negative electrode 510b via the retention member 541 is cancelled, both the electrodes 510a and 510b are switched to a non-short-circuit state. In a state after the external electrode terminal 310 is detached from the electrode terminal 510, since the retention member 541 is retained in the broken state, electrical connection between both the electrodes 510a and 510b via the retention member 541 remains in the cancelled state, and thus, the non-short-circuit state of the electrode terminal 510 is retained.

In accordance with an exemplary embodiment, since the retention member 541 is configured to be a sheet-like member, the broken retention member 541 is stored in a clearance between the electrode terminal 510 and the external electrode terminal 310. Therefore, there is no need to perform processing of the electrode terminal 510 and to provide an accommodation space as in the above-described embodiment and Modification Example 1.

As described above, according to the diagnostic imaging catheter 100 in the present embodiment, the switch unit 540 has the retention member 541 which retains the short-circuit state of both the electrodes 510a and 510b in a state before the external electrode terminal 310 is inserted into the electrode terminal 510 and retains a state where the short-circuit state of both the electrodes 510a and 510b is cancelled in a state after the external electrode terminal 310 is detached from the electrode terminal 510. Accordingly, until the diagnostic imaging catheter 100 is connected to the external electrode terminal 310 after the shipment, the electrode terminal 510 electrically connected to the ultrasound transducer 145a can be kept in a short-circuit state. Therefore, depolarization of the ultrasound transducer 145a is suppressed, and a piezoelectric effect from polarization can be retained. In addition, once the external electrode terminal 310 is connected to the electrode terminal 510, and then, the external electrode terminal 310 is detached thereafter, the non-short-circuit state can be retained.

In addition, the retention member 541 has the conductive sheet material which can cause the positive electrode 510a and the negative electrode 510b to be electrically connected to each other, and the conductive sheet material is configured to be able to be broken in response to insertion of the external electrode terminal 310 with respect to the electrode terminal 510. In this manner, both the electrodes 510a and 510b can be switched from a short-circuit state to a non-short-circuit state by adopting a simple structure using the conductive sheet material.

Hereinbefore, the diagnostic imaging catheter according to the present disclosure has been described through the embodiment and multiple modification examples. However, the present disclosure is not limited to only the configurations described in the embodiment and can be suitably changed based on Claims.

For example, as a diagnostic imaging catheter which is a target to be applied with the above-described diagnostic imaging catheter, the diagnostic imaging catheter used in the intra-vascular ultrasound diagnostic method (IVUS) is exemplified. However, the diagnostic imaging catheter which is a target to be applied is not limited thereto. For example, the above-described diagnostic imaging catheter can be applied to a hybrid-type (dual-type) diagnostic imaging catheter which can be used in both the intra-vascular ultrasound diagnostic method and an optical coherence tomography (OCT) diagnostic method.

In addition, description has been given regarding a case where the cylindrical portion of the electrode terminal (the coaxial receptacle) is the negative electrode and the portion extending along the central axis in the cylindrical portion is the positive electrode. On the contrary, the cylindrical portion of the electrode terminal (the coaxial receptacle) may be the positive electrode and the portion extending along the central axis in the cylindrical portion may be the negative electrode.

In addition, description has been given regarding a case where the electrode terminal of the diagnostic imaging catheter is configured to be the coaxial receptacle (the male connector unit) and the external electrode terminal of the external apparatus is configured to be the coaxial plug (the female connector unit). On the contrary, the electrode terminal may be configured to be the coaxial plug (the female connector unit) and the external electrode terminal may be configured to be the coaxial receptacle (the male connector unit).

In addition, the electrode terminal may be not only the coaxial plug but may also be a flat-type plug, for example, in which a positive electrode and a negative electrode having flat plate shapes extend while facing each other with a clearance provided therebetween.

In addition, for example, the movable direction of the conductive member is not limited to the extending direction of the electrode terminal or the direction intersecting the extending direction of the electrode terminal. For example, the conductive member may be configured to be movable in both directions of the extending direction of the electrode terminal and the direction intersecting the extending direction of the electrode terminal.

In addition, in Modification Example 2, description has been given regarding a case where the retention member functions as the conductive member. However, for example, the retention member and the conductive member may be configured to be separate members.

In addition, in Modification Example 2, description has been given regarding a case where the retention member is configured to be formed of a conductive sheet. However, for example, the retention member may be configured to be formed of a conductive wire or the like coming into contact with the positive electrode and the negative electrode and may be configured to be broken in response to insertion of the external electrode terminal.

In addition, for example, the sheet-like retention member according to Modification Example 2 can be combined with the configurations according to the above-described embodiment and Modification Example 1.

The detailed description above describes a diagnostic imaging catheter. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic imaging catheter comprising:
a sheath that is inserted into a body-cavity in a living body;
an ultrasound transducer that is inserted into the sheath and is configured to transmit and receive an ultrasound wave;
a connector unit that is provided in a proximal portion of the sheath;
an electrode terminal that is disposed inside the connector unit and is electrically connected to an external electrode terminal included in an external apparatus which transmits and receives an electrical signal with respect to the ultrasound transducer;
a conductive member that is provided inside the connector unit, the conductive member electrically connected to a positive electrode and a negative electrode of the electrode terminal, and which causes both the positive and the negative electrodes to be in a short-circuit state; and
a switch unit that is configured to cancel the short-circuit state of both the positive and the negative electrodes caused by the conductive member, in response to connection between the electrode terminal and the external electrode terminal.

2. The diagnostic imaging catheter according to claim 1,
wherein the switch unit has a biasing member which causes biasing force in a direction approaching any one electrode side between both the positive and the negative electrodes to be applied to the conductive member, and
wherein the switch unit is configured to be able to cancel the short-circuit state of both the positive and the negative electrodes as the conductive member is separated from the one electrode against the biasing force of the biasing member when the external electrode terminal is inserted into the electrode terminal.

3. The diagnostic imaging catheter according to claim 2,
wherein the conductive member is supported in a movable state by the biasing member in an extending direction of the electrode terminal and/or in a direction intersecting the extending direction of the electrode terminal.

4. The diagnostic imaging catheter according claim 1,
wherein the switch unit has a retention member which retains the short-circuit state of both the electrodes in a state before the external electrode terminal is inserted into the electrode terminal and retains a state where the short-circuit state of both the electrodes is cancelled in a state after the external electrode terminal is detached from the electrode terminal.

5. The diagnostic imaging catheter according to claim 4,
wherein the retention member has a conductive sheet material which causes both the electrodes to be electrically connected to each other, and
wherein the conductive sheet material is configured to be able to be broken in response to insertion of the external electrode terminal with respect to the electrode terminal.

6. The diagnostic imaging catheter according to claim 2, wherein the biasing member includes a support portion which is fixed to an inner wall of the negative electrode on a proximal side and turnably supports a proximal side of the conductive member, and a spring which is provided between the inner wall of the negative electrode and the conductive member.

7. The diagnostic imaging catheter according to claim 2, wherein the conductive member is a spherical member, the spherical member configured to be disposed in a fitting groove of the electrode terminal, and wherein the conductive member is configured to slide between the positive electrode and the negative electrode.

8. A diagnostic imaging catheter comprising:
a sheath;
an ultrasound transducer that is inserted into the sheath and is configured to transmit and receive an ultrasound wave;
a connector unit arranged in a proximal portion of the sheath;
an electrode terminal disposed inside the connector unit and configured to e electrically connected to an external electrode terminal, which transmits and receives an electrical signal with respect to the ultrasound transducer;
a conductive member inside the connector unit, the conductive member electrically connected to a positive electrode and a negative electrode of the electrode terminal, and which causes both the positive and the negative electrodes to be in a short-circuit state; and
a switch unit that is configured to cancel the short-circuit state of both the positive and the negative electrodes caused by the conductive member, in response to connection between the electrode terminal and the external electrode terminal.

9. The diagnostic imaging catheter according to claim 8,
wherein the switch unit has a biasing member which causes biasing force in a direction approaching any one electrode side between both the positive and the negative electrodes to be applied to the conductive member, and
wherein the switch unit is configured to be able to cancel the short-circuit state of both the positive and the negative electrodes as the conductive member is separated from the one electrode against the biasing force of the biasing member when the external electrode terminal is inserted into the electrode terminal.

10. The diagnostic imaging catheter according to claim 9,
wherein the conductive member is supported in a movable state by the biasing member in an extending direction of the electrode terminal and/or in a direction intersecting the extending direction of the electrode terminal.

11. The diagnostic imaging catheter according claim 8,
wherein the switch unit has a retention member which retains the short-circuit state of both the electrodes in a state before the external electrode terminal is inserted into the electrode terminal and retains a state where the short-circuit state of both the electrodes is cancelled in a state after the external electrode terminal is detached from the electrode terminal.

12. The diagnostic imaging catheter according to claim 11,
wherein the retention member has a conductive sheet material which causes both the electrodes to be electrically connected to each other, and
wherein the conductive sheet material is configured to be able to be broken in response to insertion of the external electrode terminal with respect to the electrode terminal.

13. The diagnostic imaging catheter according to claim 9, wherein the biasing member includes a support portion which is fixed to an inner wall of the negative electrode on a proximal side and turnably supports a proximal side of the conductive member, and a spring which is provided between the inner wall of the negative electrode and the conductive member.

14. The diagnostic imaging catheter according to claim 9, wherein the conductive member is a spherical member, the spherical member configured to be disposed in a fitting groove of the electrode terminal, and wherein the conductive member is configured to slide between the positive electrode and the negative electrode.

15. A method comprising:
inserting a diagnostic catheter into a blood vessel of a human body, the diagnostic catheter including a sheath that is inserted into a body-cavity in a living body, an ultrasound transducer that is inserted into the sheath and is able to transmit and receive an ultrasound wave, a connector unit that is provided in a proximal portion of the sheath, an electrode terminal that is disposed inside the connector unit and is electrically connected to an external electrode terminal included in an external apparatus which transmits and receives an electrical signal with respect to the ultrasound transducer, a conductive member that is provided inside the connector unit, is electrically connected to a positive electrode and a negative electrode of the electrode terminal, and causes both the electrodes to be in a short-circuit state, and a switch unit that is configured to be able to cancel the short-circuit state of both the electrodes caused by the conductive member, in response to connection between the electrode terminal and the external electrode terminal.

16. The method according to claim 15, comprising:
connecting the electrode terminal to the external electrode terminal; and
cancelling the short-circuit state of both the electrodes.
